(19) United States
(12) Patent Application Publication
(10) Pub. No.: US 2010/0096566 A1
(43) Pub. Date: Apr. 22, 2010

(54) Reducing Line Edge Roughness by Particle Beam Exposure

(76) Inventors: Robert Bristol, Portland, OR (US); David Ruzic, Urbana, IL (US); Corey Struck, Sidell, IL (US)

Correspondence Address: COOL PATENT, P.C.;c/o CPA Global, P.O. BOX 52050, MINNEAPOLIS, MN, 55402, US

(21) Appl. No.: 12/254,756

(22) Filed: Oct. 20, 2008

(51) Int. Cl. A61N 5/00

(52) U.S. Cl. 250/492.2

(57) ABSTRACT

Reducing line edge roughness by particle beam exposure is generally described. In one example, a method includes forming one or more line structures on a surface of a semiconductor substrate, aligning the one or more line structures to a beam path of a particle beam such that particles of the particle beam travel within 45 degrees of parallel to a lengthwise direction of the one or more line structures, and exposing the one or more line structures to the particle beam to reduce line edge roughness of the one or more line structures wherein an incident angle of the particle beam to the surface of the semiconductor substrate is between about 45 degrees and about 90 degrees, where 0 degrees is normal to the surface of the semiconductor substrate.

### BACKGROUND

Generally, line edge roughness of integrated circuit (IC) structures may not decrease proportionally with scaled dimensions such as, for example, a shrinking critical dimension (CD). Line edge roughness may negatively affect performance of a semiconductor device.

### BRIEF DESCRIPTION OF THE DRAWINGS

Embodiments disclosed herein are illustrated by way of example, and not by way of limitation, in the figures of the accompanying drawings in which like reference numerals refer to similar elements and in which:

FIG. 1 is a schematic of a system for reducing line edge roughness by particle beam exposure, according to but one embodiment;

FIG. 2 is a plan view and cross-section elevation view schematic of one or more line structures having line edge roughness on a semiconductor substrate, according to but one embodiment;

FIG. 3 is a schematic of a semiconductor substrate comprising one or more line structures, according to but one embodiment;

FIG. 4 is a schematic of a system and method for reducing line edge roughness, according to but one embodiment; and

FIG. 5 is a process flow diagram of a method for reducing line edge roughness, according to but one embodiment.

For simplicity and/or clarity of illustration, elements illustrated in the figures have not necessarily been drawn to scale. For example, the dimensions of some of the elements may be exaggerated relative to other elements for clarity. Further, if considered appropriate, reference numerals have been repeated among the figures to indicate corresponding and/or analogous elements.

### DETAILED DESCRIPTION

Embodiments of reducing line edge roughness by particle beam exposure are described herein. In the following description, numerous specific details are set forth to provide a thorough understanding of embodiments disclosed herein. One skilled in the relevant art will recognize, however, that the embodiments disclosed herein can be practiced without one or more of the specific details, or with other methods, components, materials, and so forth. In other instances, well-known structures, materials, or operations are not shown or described in detail to avoid obscuring aspects of the specification.

Reference throughout the specification to “one embodiment” or “an embodiment” means that a particular feature, structure or characteristic described in connection with the embodiment is included in at least one embodiment. Thus, appearances of the phrases “in one embodiment” or “in an embodiment” in various places throughout the specification are not necessarily referring to the same embodiment. Furthermore, the particular features, structures or characteristics may be combined in any suitable manner in one or more embodiments.

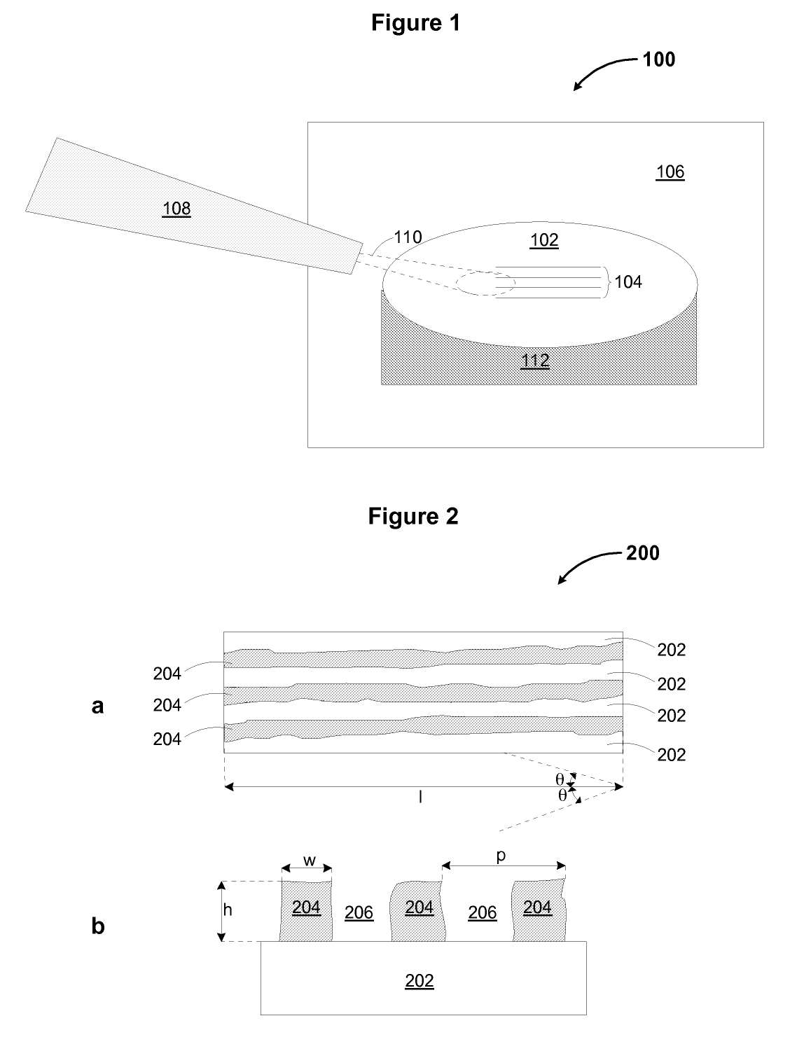

FIG. 1 is a schematic of a system for reducing line edge roughness by particle beam exposure, according to but one embodiment. In an embodiment, system 100 comprises a semiconductor substrate 102, one or more line structures 104, vacuum chamber 106, particle beam source 108, a particle beam path 110, and a substrate holder 112, coupled as shown.

Semiconductor substrate 102 may comprise a variety of substrates including semiconductor wafers of various sizes. In an embodiment, semiconductor substrate 102 comprises a semiconductor wafer having a diameter between about 100 millimeter (mm) and about 450 mm and a thickness between about 500 microns and 1000 microns. One or more dies comprising one or more discrete integrated circuit (IC) devices having nano- and micro-scale structures may be formed on the surface of the semiconductor substrate 102 in a series of process operations. In an embodiment, semiconductor substrate 102 comprises silicon, germanium, group III-V semiconductor materials, group II-VI semiconductor materials, dopants or other impurities, other suitable semiconductor materials, or combinations thereof

One or more line structures 104 may be formed on a surface of the semiconductor substrate 102 including on intervening layers or structures formed on the surface of the semiconductor substrate 102. One or more line structures 104 may include, for example, gate structures or interconnect structures, or combinations thereof, of an IC device. Line edge roughness of the one or more line structures 104 may not decrease proportionally with scaled dimensions as IC devices shrink resulting in decreased device performance. For example, line edge roughness may result in shorts, voids, or other defects. Line edge roughness and one or more line structures 104 are further described with respect to FIG. 2.

System 100 may include a particle beam source 108 disposed to provide a particle beam having a particle beam path 110. In an embodiment, the particle beam source 108 comprises an ion source. The ion source may comprise a range of energies including a range from about 50 electron-volts (eV) to several keV of energy. In an embodiment, particle beam source 108 provides a beam comprising a current density from about 10 milliamps (mA) per cm² to about 1000 mA per cm². An area of a particle beam from particle beam source 108 may cover an entire surface of semiconductor substrate 102 or may cover only a portion of the surface of the semiconductor substrate 102. Particle beam source 108 may provide a particle beam comprising a total current of fractions of an Amp to 10's of Amps. In an embodiment, a particle beam source 108 provides a particle beam comprising a current density between about 100 mA/cm² to about 200 mA/cm², a diameter of about 3 cm, and a total current of about 5 Amps (A). Subject matter is not limited in this regard and may include other ranges in other embodiments.

Particle beam path 110 may represent a path of travel for particles from particle beam source 108 upon turning on the particle beam. Upon turning on the particle beam, a particle beam having particle beam path 110 is directed at the one or more line structures 104 of semiconductor substrate 102 to reduce line edge roughness. Particle beam source 108 may provide a particle beam comprising, for example, an ion beam, an atomic beam, an electron beam, or a photon beam, or combinations thereof.

In an embodiment, particle beam source 108 provides a collimated beam comprising neon (Ne) or argon (Ar) atoms or ions, or combinations thereof In another embodiment, particle beam source 108 provides a rasterized beam. A particle beam from particle beam source 108 may reduce line edge roughness by sputtering in one embodiment.

In another embodiment, particle beam source 108 provides a particle beam that is used in combination with an activated etchant to accelerate reduction of line edge roughness. Etchants may comprise, for example, halogens such as chlorine (Cl) or fluorine (Fl), or other elements such as hydrogen, or combinations thereof. In an embodiment, reactive ion etching is used in combination with exposure to particle beam to reduce line edge roughness. Particle beam may chemically activate one or more etchants. Radicals such as Cl⁻ may form that more readily bond with broken bond materials formed by particle beam exposure. Such materials may more readily be removed in combination with such etchants. Subject matter is not limited in this regard and may include other etchants in other embodiments.

A variety of particle beam exposure dosages may be selected based on a variety of factors including at least the material of the one or more line structures 104, pressure within the vacuum chamber 106, energy of the particle beam source 108, type of particles to be directed down the particle beam path 110, desired amount of sputtering, angle of approach of the particle beam path 110 to the semiconductor substrate 102, and area of coverage on the substrate 102 by the particle beam path 110. Exposure dosage may be controlled by exposure time using, for example, a shutter. In one embodiment, an atomic beam source 108 that produces about 1×10¹⁷ neon (Ne) atoms or ions per second over a beam path 110 having an area of coverage of about 25 mm on the semiconductor substrate 102, reduces line edge roughness of line structures 104 comprising carbon by about 1 nanometer during a 20 second exposure time. In an embodiment, a dose may be selected to provide sputtering on the order of about 1-10 nanometers.

In another embodiment, an example calculation to provide about a 3 nm sputtering thickness of line structures 104 comprising carbon using a particle beam comprising argon (Ar) atoms or ions at 500 eV is provided. A 3 nm sputtering thickness comprises about 15 carbon atoms. Therefore, about 4×10¹⁶ carbon atoms are to be sputtered per cm² of the line structures 104. Sputter yield may approach unity at an incident angle of about 85 degrees, therefore about 4×10¹⁶ Ar atoms or ions are needed to sputter 4×10¹⁶ carbon atoms. If an exposure time of about 10 seconds (s) is desired, then about 4×10¹⁵ Ar atoms or ions per second are needed. When the particle beam is tilted in a direction away from normal to the surface of semiconductor substrate 102, the cross-sectional area of the particle beam impinging on the surface increases thereby decreasing the flux by a geometrical factor. In this example, the flux is reduced about 10-fold. Therefore the geometrical factor is about 0.1. A geometrical cross-section factor of about 0.1 requires a beam of about 4×10¹⁶ Ar atoms or ions per cm²s. Multiplying 4×10¹⁶ Ar atoms or ions per cm²s by an elementary charge of 1.6×10⁻¹⁹ coulombs (C) gives an implied current of about 6.4 milliamps (mA) per cm².

Particle beam source 108 may provide a photon beam. In an embodiment, a photon beam comprises a wavelength significantly less than a critical dimension to preferentially deposit energy to reduce line edge roughness. In an embodiment, particle beam source 108 provides a photon beam having a wavelength less than about 10 nanometers (nm). In an embodiment, a photon beam is combined with a photo-activated etchant as described above to accelerate reduction of line edge roughness.

In an embodiment, particle beam source 108 provides a particle beam comprising an electron beam. An electron beam may be used to break bonds associated with line edge roughness of the one or more line structures 104. Such broken bonds may facilitate reduction of line edge roughness by allowing etchants to more easily remove the broken bond material or making sputtering by atomic or ion beam easier, or combinations thereof. In an embodiment, electron beam from particle beam source 108 chemically activates etchants to accelerate reduction of line edge roughness.

In an embodiment, the particle beam comprises an ion beam neutralized by an electron beam. An ion beam may allow more energy per atom than an atomic beam. However, the ion beam may have a beam path 110 that turns towards the semiconductor substrate 102 depending on the electric potential of the semiconductor substrate 102.

A voltage source may be coupled with vacuum chamber 106, substrate holder 112, or other elements of system 100 to provide a bias to the semiconductor substrate 102. A bias potential may aid in controlling the energy of a particle beam such as an ion beam from particle beam source 108 or attracting secondary electrons produced by particle beam exposure.

System 100 may include a vacuum chamber 106. In an embodiment, vacuum chamber 106 provides sufficient vacuum such that particles may travel according to a particle beam path 110 towards the surface of the semiconductor substrate 102 without significant scattering. In an embodiment vacuum chamber 106 provides vacuum sufficient to allow a mean free path of particles from particle beam source 108 of about 5 centimeters (cm) per milliTorr (mTorr) or more. In another embodiment, vacuum chamber 106 is capable of providing a pressure of about 1×10⁻³ Torr to about 1×10⁻⁶ Torr. In yet another embodiment, vacuum chamber 106 is capable of providing a pressure less than about 1×10⁻³ Torr. Subject matter is not limited in this regard and other pressures may be used in other embodiments.

In an embodiment, vacuum chamber 106 comprises an etch chamber. System 100 may be implemented, for example, in existing etch equipment by adding a beam source 108 to an etch chamber 106. In an embodiment, reducing line edge roughness 108 by particle beam exposure is part of an etch recipe. One or more etchants may be used in combination with particle beam exposure to reduce line edge roughness of the one or more line structures 104 as described above. Implementing system 100 within etch equipment may at least include the benefits of reduced cost, fewer process operations, acceleration of reducing line edge roughness by combining particle beam exposure with etchants, and having a voltage source for wafer biasing within existing equipment. In an embodiment, system 100 is part of other existing semiconductor fabrication equipment or, in another embodiment, may comprise stand-alone equipment.

System 100 may include a substrate holder 112 to hold a semiconductor substrate 102. In an embodiment, substrate holder 112 comprises a stage, a chuck, a platform, a robot, any other suitable substrate holder, or combinations thereof Substrate holder 112 may comprise means to hold the semiconductor substrate 102 securely, using, for example, vacuum, or a mechanical securing device, or combinations thereof Substrate holder 112 may comprise means to rotate the semiconductor substrate 102 about an axis normal to the surface of the semiconductor substrate 102, the axis being at or near the center of the semiconductor substrate 102. In an embodiment, substrate holder 112 comprises means to align the semiconductor substrate 102 with the particle beam path 110 of particle beam source 108 in accordance with orientations described herein.

In an embodiment, system 100 includes a vacuum chamber 106, a particle beam source 108 coupled with the vacuum chamber to produce a particle beam having a particle beam path 110, the particle beam to reduce line edge roughness of one or more line structures 104 formed on a surface of a semiconductor substrate 102. System 100 may further include a substrate holder 112 to hold the semiconductor substrate 102 wherein an incident angle of the particle beam path 110 to the surface of the semiconductor substrate 102 is between about 45 degrees and about 90 degrees, where 0 degrees is normal to the surface of the semiconductor substrate 102. In another embodiment, system 100 includes a substrate holder 112 to hold the semiconductor substrate 102 wherein the particle beam path 110 is directed within about 45 degrees of parallel to a lengthwise direction of the one or more line structures 104. Such orientations may be further described with respect to at least FIGS. 2-4.

FIG. 2 is a plan view and cross-section elevation view schematic of one or more line structures having line edge roughness on a semiconductor substrate, according to but one embodiment. FIG. 2a may provide a plan view of one ore more line structures 204 having line edge roughness on a semiconductor substrate 202 and FIG. 2a may provide a cross-section elevation view schematic of one or more line structures 204 having line edge roughness on a semiconductor substrate 202.

In an embodiment according to FIG. 2a, an article of manufacture 200 comprises one or more line structures 204 coupled with a semiconductor substrate 202. FIG. 2a may depict the one or more line structures 204 from a perspective normal to the surface of the semiconductor substrate 202. One or more line structures 204 may be formed on or into a surface of semiconductor substrate 202 by one or more semiconductor processes including, for example, photolithography, etching, thermal treatments, film deposition, or planarization, or combinations thereof.

One or more line structures 204 may include a variety of structures. In an embodiment, one or more line structures 204 include, for example, gate structures, or interconnect structures, or combinations thereof. One or more line structures 204 may also comprise a variety of materials. In an embodiment, the one or more line structures 204 include photosensitive material, dielectric material, electrode material, other materials associated with semiconductor fabrication, or any suitable combination thereof. For example, one or more line structures 204 that may benefit from exposure to a particle beam to reduce line edge roughness may include photoresist structures 204 prior to etching or one or more line structures 204 formed into an underlying material after etching. Claimed subject matter is not limited in this regard and may include other structures or materials in other embodiments.

One or more line structures 204 may comprise line edge roughness. Line edge roughness may become increasingly problematic as critical dimensions (CD) of line structures 204 such gate or metal lines continue to decrease. In an embodiment, line edge roughness is a measurement of roughness of an edge of a line structure 204. In one embodiment, a three-sigma standard deviation measurement of roughness provides a line edge roughness of about 4 nm along a 500 nm length, l, of line structure 204 having a CD of about 50 nm. A line CD of 50 nm may correlate with a width, w, of about 50 nm as depicted in FIG. 2b.

Line width roughness may similarly be a measurement of roughness of line structures 204 performed by measuring line width CD at various places and determining a standard deviation with three-sigma confidence. Line width roughness may be about equal to line edge roughness multiplied by the square root of two. In an embodiment, exposure by particle beam as described herein reduces line width roughness. Particle beam exposure may reduce low frequency line width roughness.

In an embodiment according to FIG. 2a, θ is an angle along the plane of the surface of the semiconductor substrate 102, 202 that represents a deviation of the beam path 110 from the lengthwise direction, l, of the one or more line structures 204. In an embodiment, a particle beam path 110 is directed within an angle, θ, of parallel to a lengthwise direction, l, of the one or more line structures 204. The lengthwise direction may be in direction, l, indicated by the arrow in FIG. 2a. In an embodiment, θ is less than about 45 degrees. In another embodiment, θ is less than about 5 degrees. In other words, particle beam path 110 may be directed within about 45 degrees of parallel to a lengthwise direction of the one or more line structures 204 in one embodiment and within about 5 degrees of parallel to a lengthwise direction of the one or more line structures 204 in another embodiment. In yet another embodiment, semiconductor substrate 102 and beam path 110 are aligned such that the beam path 110 is substantially directed lengthwise, l, along the one or more line structures 204. One or more line structures 204 may be analogous to one or more line structures 104.

In an embodiment according to FIG. 2b, an article of manufacture 200 comprises one or more line structures 204 coupled with a semiconductor substrate 202, wherein regions between the one or more line structures 204 define one or more trench structures 206. FIG. 2b may be a cross-section depiction of FIG. 2a along a direction perpendicular to the lengthwise direction, l, of the one or more line structures 204. FIG. 2a may depict the one or more line structures 204 from a perspective substantially parallel to the surface of the semiconductor substrate 202 wherein the lengthwise direction, l, of FIG. 2a goes in and out of the page in FIG. 2b.

The one or more line structures 204 may comprise a variety of dimensions. In an embodiment, the one or more line structures comprise a pitch p, a width w, and a height h, as indicated. Pitch, p, may be a distance between repeating structures. In an embodiment, one or more line structures comprise a pitch, p, less than about 100 nm, a width, w, that is less than the pitch, a height, h, that is greater than the width, and a length, l, that is greater than the pitch. Claimed subject matter is not limited in this regard and other dimensions may be used in other embodiments. In an embodiment, the width, w, defines a line CD. A line CD may comprise a width less than about 50 nm in an embodiment. In another embodiment, the height, h, is greater than about two times the width, w.

In an embodiment, exposure to a particle beam as described herein reduces spatial wavelength components of line edge roughness on the order of about two times the pitch, p, or greater. Low frequency roughness on this order may impact device performance more than high frequency roughness.

In one or more embodiments, substrate 202 and one or more line structures 204 may comprise identical materials although a boundary appears to separate the structures in FIG. 2b. For example, one or more line structures 204 may be materially and structurally contiguous with semiconductor substrate 202 where one or more line structures 204 are formed by recessing regions 206 of a substrate comprising regions 202 and 204 prior to recessing. Substrate 202 and one or more line structures 204 may comprise different materials in other embodiments.

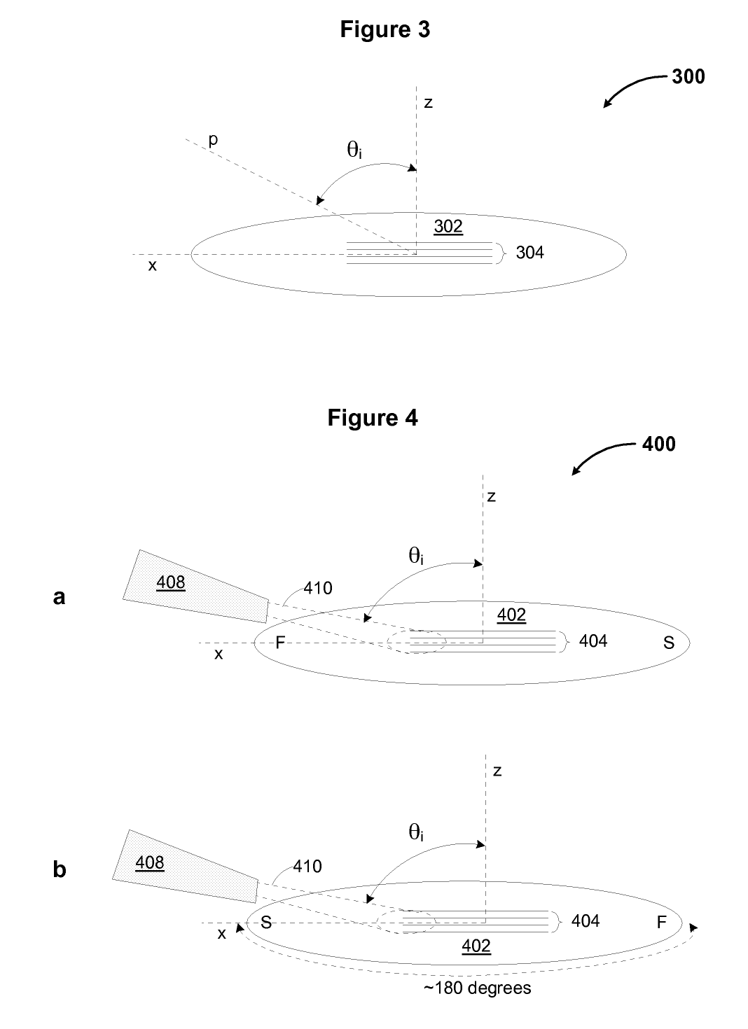

An article of manufacture 200 may comprise a semiconductor substrate 202 and one or more line structures 204 wherein the one or more line structures 204 have reduced line edge roughness as a result of exposure to a particle beam comprising an incident angle (θᵢ of FIG. 3) between about 45 degrees and about 90 degrees to the surface of the semiconductor substrate 202, where 0 degrees is normal to the surface of the semiconductor substrate 202 and wherein the particle beam is directed within about 45 degrees (θ of FIG. 2a) of parallel to the lengthwise, l, direction of the one or more line structures 204. The one or more line structures 204 may comprise one or more gate structures, or one or more interconnect structures, or combinations thereof, of an integrated circuit device.

FIG. 3 is a schematic of a semiconductor substrate comprising one or more line structures, according to but one embodiment. In an embodiment, an article of manufacture 300 includes a semiconductor substrate 302 and one or more line structures 304, coupled as shown. Article of manufacture 300 may include embodiments already described herein with respect to FIGS. 1-2. FIG. 3 may include x-axis, z-axis, beam path, p, and incident angle, θᵢ, to aid in understanding orientation aspects of subject matter described herein.

In an embodiment, x-axis lies on a plane that is substantially parallel with a surface of semiconductor substrate 302 upon which one or more line structures 304 are formed. The z-axis may be substantially normal to the x-axis in direction the indicated. A beam path, p, may lie on a plane formed by the x-axis and the z-axis where an incident angle, θᵢ, defines an angle between the z-axis and the beam path, p. Beam path, p, may be a particle beam path analogous to beam path 110 of FIG. 1.

In an embodiment, an incident angle, θᵢ, of the particle beam path, p, to the surface of the semiconductor substrate 302 is between about 45 degrees and about 90 degrees, where 0 degrees is normal to the surface of the semiconductor substrate. In another embodiment, the incident angle is between about 65 degrees and about 85 degrees, where 0 degrees is normal to the surface of the semiconductor substrate 302 in the direction of the z-axis. An incident angle between about 65 degrees and about 85 degrees may provide a higher sputtering yield for beam path, p, than for an incident angle, θᵢ, that is less than about 65 degrees.

FIG. 4 is a schematic of a system and method for reducing line edge roughness, according to but one embodiment. In an embodiment, system 400 for reducing line edge roughness includes a semiconductor substrate 402, one or more line structures 404, particle beam source 408, and particle beam path 410, coupled as shown. FIG. 4a may depict a first particle beam exposure and FIG. 4b may depict a second particle beam exposure. The x-axis, z-axis, and orientation markers F and S may aid in understanding orientation aspects of subject matter described herein. The x-axis and z-axis may be analogous to x-axis and z-axis of FIG. 3. Orientation markers F and S may be used to distinguish the relative position of semiconductor substrate 402 in a first particle beam exposure according to FIG. 4a and a second particle beam exposure according to FIG. 4b.

In an embodiment according to FIG. 4a, particle beam path 410 and the semiconductor substrate 402 may be aligned with respect to one another such that the particle beam path 410 is directed within about 45 degrees of parallel to the lengthwise direction of the one or more line structures 404 to provide a first particle beam exposure. Exposing the one or more line structures to a particle beam that follows particle beam path 410 of FIG. 4a may define a first exposure. Particle beam path 410 and the semiconductor substrate 402 may be aligned with respect to one another by any suitable means, including rotating the semiconductor substrate 402, moving the beam source 408 and beam path 410, or suitable combinations thereof.

In an embodiment according to FIG. 4b, semiconductor substrate 402 may be rotated about 180 degrees relative to FIG. 4a. Semiconductor substrate 402 may be rotated about the z-axis such that the F and S orientation markers are in an opposite orientation compared with FIG. 4a. The particle beam path 408 and the semiconductor substrate 402 may be aligned with respect to one another such that the particle beam path 408 is directed within about 45 degrees of parallel to the lengthwise direction of the one or more line structures 404 to provide a second particle beam exposure wherein the second particle beam exposure approaches the semiconductor substrate 402 from a direction substantially opposite to the first particle beam exposure in at least one dimension. For example, the second particle beam exposure may approach the semiconductor substrate 402 from a direction substantially opposite to the first particle beam exposure in at least the dimension denoted by axis-x.

Other methods of aligning particle beam path 410 and semiconductor substrate 402 to provide a first particle beam exposure and a second particle beam exposure may be used, including moving the particle beam source 408, the particle beam path 410, the semiconductor substrate 402, or combinations thereof. In an embodiment, the first particle beam exposure and the second particle beam exposure comprise a similar incident angle, θᵢ, but subject matter is not limited in this regard.

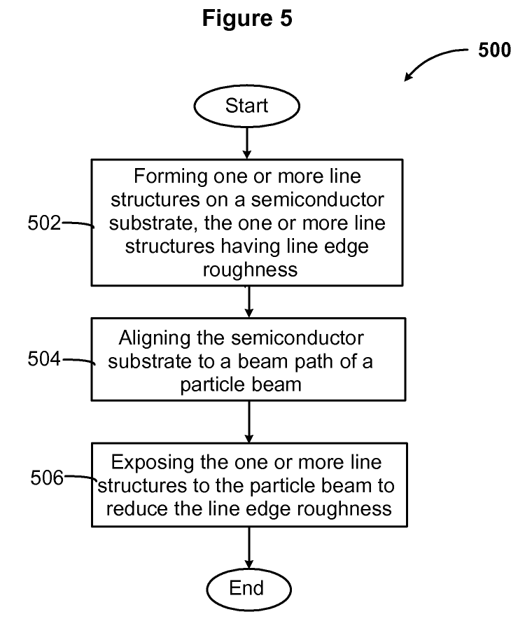

FIG. 5 is a process flow diagram of a method for reducing line edge roughness, according to but one embodiment. In an embodiment, method 500 includes forming one or more line structures on a semiconductor substrate, the one or more line structures having line edge roughness at box 502, aligning the semiconductor substrate to a beam path of a particle beam at box 504, and exposing the one or more line structures to the particle beam to reduce the line edge roughness.

Forming one or more line structures on a semiconductor substrate 502 may be accomplished in a variety of ways. In an embodiment, forming one or more line structures on a semiconductor substrate 502 is accomplished using one or more semiconductor processes including, for example, photolithography, etching, thermal treatments, film deposition, or planarization, or combinations thereof. In an embodiment, forming one or more line structures on a surface of a semiconductor substrate 502 includes forming one or more line structures having a pitch less than about 100 nm, a width that is less than the pitch, a height that is greater than the width, and a length that is greater than the pitch.

Forming one or more line structures on a surface of a semiconductor substrate 502 may include forming one or more gate structures, or one or more interconnect structures, or combinations thereof. In an embodiment, the one or more line structures are formed 502 in photosensitive material. In such embodiment, method 500 may further include etching the one or more line structures into the semiconductor substrate to form one or more line structures of an integrated circuit (IC) device. Such method 500 may provide one or more line structure of the IC device that have reduced line edge roughness compared with one or more line structures that are not exposed to the particle beam.

Benefits of exposing the one or more line structures to the particle beam 506 to reduce line edge roughness may apply to one or more line structures formed 502 using a variety of materials. In another embodiment, forming one or more line structures on a semiconductor device 502 comprises forming one or more line structures using materials other than photosensitive material, such as dielectric material, electrode material, semiconductor material, or other material associated with semiconductor fabrication, or combinations thereof.

Method 500 may further include aligning the semiconductor substrate to a beam path of a particle beam 504. In an embodiment, method 500 includes aligning the one or more line structures to a beam path of a particle beam 504 such that particles of the particle beam travel in a direction within 45 degrees of parallel to a lengthwise direction of the one or more line structures. In another embodiment, aligning the one ore more line structures to the beam path of the particle beam 504 comprises aligning the particle beam to travel within about 5 degrees of parallel to the lengthwise direction of the one or more line structures.

Method 500 may further include exposing the one or more line structures to the particle beam to reduce line edge roughness 506. In an embodiment, exposing the one or more line structures to the particle beam 506 comprises using an incident angle of the particle beam to the surface of the semiconductor substrate that is between about 45 degrees and about 90 degrees, where 0 degrees is normal to the surface of the semiconductor substrate. In another embodiment, exposing the one or more line structures to the particle beam 506 comprises using an incident angle of the particle beam to the surface of the semiconductor substrate that is between about 65 degrees and about 85 degrees, where 0 degrees is normal to the surface of the semiconductor substrate.

Exposing the one or more line structures to the particle beam 506 may be performed at a pressure between about 1×10⁻³ Torr and about 1×10⁻⁶ Torr. In an embodiment, particle beam exposure 506 is performed at a pressure less than about 1×10⁻³ Torr. Subject matter is not limited in this regard and other pressures may be used in other embodiments. Method 500 may further include pumping down a vacuum chamber to provide sufficient vacuum for particle beam exposure of semiconductor substrate 506.

In an embodiment, exposing the one or more line structures to the particle beam 506 comprises exposing the one or more line structures to an ion beam, an atomic beam, an electron beam, or a photon beam, or combinations thereof In another embodiment, exposing the one or more line structures to the particle beam 506 comprises exposing the one or more line structures to a collimated beam comprising neon (Ne) or argon (Ar), or combinations thereof

A variety of techniques may be used to expose the one or more line structures to the particle beam 506. To increase uniformity of reduction of line edge roughness, exposing the one or more line structures to the particle beam 506 may include exposing the one or more line structures to the particle beam to define a first exposure, rotating the semiconductor substrate about 180 degrees relative to the particle beam source, and exposing the one or more line structures to the particle beam to define a second exposure wherein the particle beam of the first exposure and the particle beam of the second exposure approach the semiconductor substrate from substantially opposite directions in at least one dimension. Other techniques to accomplish such exposure may be employed in other embodiments. For example, particle beam source may be moved in another embodiment, or combinations of moving the particle beam source and semiconductor substrate may be used.

Exposing one or more line structures to the particle beam 506 may reduce long spatial wavelength components of the line edge roughness. In an embodiment, exposing the one or more line structures to the particle beam 506 reduces spatial wavelength components on the order of about two times the pitch or greater.

Method 500 may further include applying one or more etchants to the one or more line structures while exposing the one or more line structures to the particle beam. One or more etchants in combination with the particle beam exposure may accelerate reducing line edge roughness of the one or more line structures.

A product fabricated by method 500 described herein is also disclosed. A product may include an article of manufacture including a semiconductor substrate and one or more line structures of an integrated device. Method 500 may include embodiments already described herein with respect to FIGS. 1-4.

Various operations may be described as multiple discrete operations in turn, in a manner that is most helpful in understanding the claimed subject matter. However, the order of description should not be construed as to imply that these operations are necessarily order dependent. In particular, these operations may not be performed in the order of presentation. Operations described may be performed in a different order than the described embodiment. Various additional operations may be performed and/or described operations may be omitted in additional embodiments.

The above description of illustrated embodiments, including what is described in the Abstract, is not intended to be exhaustive or to limit to the precise forms disclosed. While specific embodiments and examples are described herein for illustrative purposes, various equivalent modifications are possible within the scope of the description, as those skilled in the relevant art will recognize.

These modifications can be made in light of the above detailed description. The terms used in the following claims should not be construed to limit the scope to the specific embodiments disclosed in the specification and the claims. Rather, the scope of the embodiments disclosed herein is to be determined by the following claims, which are to be construed in accordance with established doctrines of claim interpretation.

What is claimed is:

1. A method comprising:
forming one or more line structures on a surface of a semiconductor substrate;
aligning the one or more line structures to a beam path of a particle beam such that particles of the particle beam travel within 45 degrees of parallel to a lengthwise direction of the one or more line structures; and
exposing the one or more line structures to the particle beam to reduce line edge roughness of the one or more line structures wherein an incident angle of the particle beam to the surface of the semiconductor substrate is between about 45 degrees and about 90 degrees, where 0 degrees is normal to the surface of the semiconductor substrate.

2. A method according to claim 1 wherein exposing the one or more line structures to the particle beam comprises using an incident angle of the particle beam to the surface of the semiconductor substrate that is between about 65 degrees and about 85 degrees, where 0 degrees is normal to the surface of the semiconductor substrate.

3. A method according to claim 1 wherein exposing the one or more line structures to the particle beam is performed at a pressure less than about 1×10⁻³ Torr.

4. A method according to claim 1 wherein exposing the one or more line structures to the particle beam comprises exposing the one or more line structures to an ion beam, an atomic beam, an electron beam, or a photon beam, or combinations thereof.

5. A method according to claim 1 wherein exposing the one or more line structures to the particle beam comprises exposing the one or more line structures to a collimated beam comprising neon (Ne) or argon (Ar), or combinations thereof.

6. A method according to claim 1 wherein aligning the one or more line structures to the beam path of the particle beam comprises aligning the particle beam to travel within about 5 degrees of parallel to the lengthwise direction of the one or more line structures.

7. A method according to claim 1 wherein exposing the one or more line structures to the particle beam comprises:
exposing the one or more line structures to the particle beam to define a first exposure;
rotating the semiconductor substrate about 180 degrees; and
exposing the one or more line structures to the particle beam to define a second exposure wherein the particle beam of the first exposure and the particle beam of the second exposure approach the semiconductor substrate from substantially opposite directions in at least one dimension.

8. A method according to claim 1 further comprising:
applying one or more etchants to the one or more line structures while exposing the one or more line structures to the particle beam to accelerate reduction of line edge roughness of the one or more line structures.

9. A method according to claim 1 wherein forming the one or more line structures on the surface of the semiconductor substrate comprises forming one or more gate structures, or one or more interconnect structures, or combinations thereof, in photosensitive material.

10. A method according to claim 9 further comprising:
etching the one or more line structures into the semiconductor substrate to form one or more line structures of an integrated circuit (IC) device wherein the one or more line structures of the IC device comprise reduced line edge roughness compared with one or more line structures that are not exposed to the particle beam.

11. A method according to claim 1 wherein forming the one or more line structures on the surface of the semiconductor substrate comprises forming the one or more line structures having a pitch less than about 100 nanometers (nm), a width that is less than the pitch, a height that is greater than the width, and a length that is greater than the pitch.

12. A method according to claim 11 wherein exposing the one or more line structures to the particle beam reduces spatial wavelength components of the line edge roughness on the order of about two times the pitch or greater.

13. A product fabricated by the method of claim 1.

14. A system comprising:
a vacuum chamber;
a particle beam source coupled with the vacuum chamber to produce a particle beam having a particle beam path, the particle beam to reduce line edge roughness of one or more line structures formed on a surface of a semiconductor substrate; and
a substrate holder within the vacuum chamber to hold the semiconductor substrate wherein an incident angle of the particle beam path to the surface of the semiconductor substrate is between about 45 degrees and about 90 degrees, where 0 degrees is normal to the surface of the semiconductor substrate, and wherein the particle beam path is directed within about 45 degrees of parallel to a lengthwise direction of the one or more line structures.

15. A system according to claim 14 wherein the incident angle is between about 65 degrees and about 85 degrees, where 0 degrees is normal to the surface of the semiconductor substrate.

16. A system according to claim 14 wherein the vacuum chamber is capable of providing a pressure less than about 1×10⁻³ Torr.

17. A system according to claim 14 wherein the particle beam comprises an ion beam, an atomic beam, an electron beam, or a photon beam, or combinations thereof.

18. A system according to claim 14 wherein the particle beam is a collimated beam comprising neon (Ne) or argon (Ar), or combinations thereof.

19. A system according to claim 14 wherein the particle beam path and the semiconductor substrate can be aligned with respect to one another such that the particle beam path is directed within about 5 degrees of parallel to the lengthwise direction of the one or more line structures to provide a first particle beam exposure.

20. A system according to claim 19 wherein the particle beam path and the semiconductor substrate can be aligned with respect to one another such that the particle beam path is directed within about 5 degrees of parallel to the lengthwise direction of the one or more line structures to provide a second particle beam exposure wherein the second particle beam exposure comprises a similar incident angle as the first particle beam exposure and wherein the second particle beam exposure approaches the semiconductor substrate from a direction substantially opposite to the first particle beam exposure in at least one dimension.

21. A system according to claim 14 wherein the vacuum chamber comprises an etch chamber.

22. A system according to claim 21 wherein one or more etchants can be used in combination with the particle beam to reduce the line edge roughness of the one or more line structures.

23. A system according to claim 14 wherein the one or more line structures comprise gate structures, or interconnect structures, or combinations thereof, the one or more line structures comprising photosensitive material, dielectric material, electrode material, semiconductor fabrication material, or combinations thereof.

24. A system according to claim 14 wherein the one or more line structures comprise a pitch less than about 100 nanometers (nm), a width that is less than the pitch, a height that is greater than the width, and a length that is greater than the pitch.

25. A system according to claim 24 wherein the particle beam reduces spatial wavelength components of the line edge roughness on the order of about two times the pitch or greater.

26. A system according to claim 14 further comprising:
a voltage source coupled with the substrate holder to provide a bias to the semiconductor substrate.

27. An apparatus comprising:
a semiconductor substrate; and
one or more line structures formed on or into a surface of the semiconductor substrate wherein the one or more line structures have reduced line edge roughness as a result of exposure to a particle beam comprising an incident angle between about 45 degrees and about 90 degrees to the surface of the semiconductor substrate, where 0 degrees is normal to the surface of the semiconductor substrate, and wherein the particle beam is directed within about 45 degrees of parallel to the lengthwise direction of the one or more line structures.

28. An apparatus according to claim 27 wherein the one or more line structures comprise one or more gate structures, or one or more interconnect structures, or combinations thereof, of an integrated circuit device.

29. An apparatus according to claim 27 wherein the one or more line structures are formed into the surface of the semiconductor substrate by etching, the one or more line structures comprising a pitch that is less than about 100 nanometers (nm), a width that is less than the pitch, a height that is greater than the width, and a length that is greater than the pitch.

30. An apparatus according to claim 27 wherein the one or more line structures have reduced line edge roughness as a result of exposure to the particle beam from a first direction and a second direction wherein the first direction and the second direction are substantially opposite to one another in at least one dimension.
